(19) United States
(12) Patent Application Publication
(10) Pub. No.: US 2026/0144966 A1
(43) Pub. Date: May 28, 2026

(54) BALLOON CATHETER

(71) Applicant: KANEKA CORPORATION, Osaka (JP)

(72) Inventors: Kazuki Matsufuji, Osaka (JP); Masahiro Kojima, Osaka (JP); Takahisa Hamabuchi, Osaka (JP)

(73) Assignee: KANEKA CORPORATION, Osaka (JP)

(21) Appl. No.: 19/424,324

(22) Filed: Dec. 18, 2025

Related U.S. Application Data
(63) Continuation of application No. PCT/JP2024/022198, filed on Jun. 19, 2024.

(30) Foreign Application Priority Data
Jun. 22, 2023 (JP) 2023-102818

(51) Int. Cl. A61M 25/10

(52) U.S. Cl. CPC A61M 25/1011; A61M 2025/1061; A61M 2210/125

(57) ABSTRACT

A balloon catheter including a shaft extending in the longitudinal direction from the proximal side toward the distal side and a plurality of balloons arranged at a distal portion of the shaft is provided. The shaft includes an inner tubular member having a lumen and a tubular member group constituted by a plurality of outer tubular members, each having a lumen, disposed on the outer side with respect to the inner tubular member and aligned in the circumferential direction of the inner tubular member. In a cross-section perpendicular to the longitudinal direction, the shaft includes a portion in which the distance (D10) between the center of a first outer tubular member and the center of a second outer tubular member is larger than the distance (D11) between the center of the first outer tubular member and the center of a third outer tubular member.

### TECHNICAL FIELD

One or more embodiments of the present invention relate to a balloon catheter.

### BACKGROUND

A stenosed site hardened by calcification or the like is formed in an inner wall of a blood vessel, thereby causing diseases such as angina pectoris and myocardial infarction. One of treatments for these diseases is angioplasty, in which a balloon catheter is used to dilate a stenosed site. Angioplasty is a minimally invasive treatment, which does not require thoracotomy such as a bypass operation, is widely performed.

There is a disease called aortic valve stenosis, in which an aortic valve becomes hard due to calcification or the like, the aortic valve becomes difficult to open, and the flow of blood is blocked. As a treatment for aortic valve stenosis, a method in which a bioprosthetic valve (artificial valve) is placed by a surgical open-chest operation and a catheter and a hardened aortic valve is replaced with the bioprosthetic valve is sometimes used.

Implanted bioprosthetic valves deteriorate over time due to calcification, wear, and the like. When an implanted bioprosthetic valve deteriorates, the bioprosthetic valve needs to be replaced. In a technique that has been studied for replacement of bioprosthetic valves, a balloon catheter having a braid or a plurality of balloon catheters is used to apply high pressure to an indwelling bioprosthetic valve to deform or break the valve, thereby dilating the lumen of the valve, and then a new bioprosthetic valve is indwelled inside the deformed or broken bioprosthetic valve by transcatheter aortic valve replacement or the like.

As a catheter that is used for dilation of hardened stenosed sites and for indwelling of bioprosthetic valves and that includes a balloon inflatable by a high pressure, for example, PTL 1 discloses a balloon catheter including a plurality of balloon members and in which a plurality of outer balloon members are disposed so as to surround the outer surface of an inner balloon member, and PTL 2 discloses a device that includes a perfusion balloon having an internal passage and a balloon disposed in the internal passage of the perfusion balloon.

### Patent Literature

PTL 1: US Patent Application Publication No. 2012/0209375

PTL 2: Japanese Unexamined Patent Application Publication (Translation of PCT Application) No. 2018-536474

A shaft of a catheter, such as the conventional catheters described in PTL 1 and PTL 2, including a plurality of balloons may be configured to have a plurality of lumens such as inflation lumens connected to respective balloons and through which a fluid to be supplied into the lumens of the balloons passes and a guidewire lumen through which a guidewire is inserted. When a shaft has a plurality of lumens, the rigidity of the shaft tends to increase due to the presence of partition walls between the lumens and an increase in the outer diameter of the shaft. When the rigidity of a shaft is high, the shaft is difficult to bend, and insertion of the shaft is difficult since the shaft is not able to sufficiently follow a lumen in a living body, such as a curved blood vessel, and may apply a physical stimulus to a tube wall of the lumen in the living body, for example, may damage the tube wall.

### SUMMARY

In consideration of the above-described circumstances, a balloon catheter including a shaft that is flexible to easily follow a lumen in a living body and that is easily inserted into a lumen in a living body is provided.

A balloon catheter according to one or more embodiments of the present invention capable of addressing the above is as follows.
[1] A balloon catheter including:
a shaft extending from a proximal side toward a distal side in a longitudinal direction; and
a plurality of balloons disposed at a distal portion of the shaft,
in which the shaft includes an inner tubular member having a lumen, and a tubular member group including a plurality of outer tubular members each having a lumen, the plurality of outer tubular members being disposed on an outer side with respect to the inner tubular member and aligned in a circumferential direction of the inner tubular member,
in which, in a cross-section perpendicular to the longitudinal direction, the tubular member group includes a first outer tubular member, a second outer tubular member disposed adjacent to the first outer tubular member in the circumferential direction of the inner tubular member, and a third outer tubular member disposed adjacent to the first outer tubular member in the circumferential direction of the inner tubular member and on a side opposite to the second outer tubular member, and
in which, in a cross-section perpendicular to the longitudinal direction, the shaft includes a portion in which a distance between a center of the first outer tubular member and a center of the second outer tubular member is larger than a distance between a center of the first outer tubular member and a center of the third outer tubular member.
[2] The balloon catheter according to [1],
in which the shaft has a distal region, which is a region on a distal side, and a proximal region, which is a region located on a proximal side with respect to the distal region, and
in which, in a cross-section in the proximal region and perpendicular to the longitudinal direction, a distance between a center of the first outer tubular member and a center of the second outer tubular member is larger than a distance between a center of the first outer tubular member and a center of the third outer tubular member.
[3] The balloon catheter according to [2], in which, in the longitudinal direction, a length of the proximal region is equal to or less than a length of the distal region.
[4] The balloon catheter according to [2] or [3], in which the inner tubular member has, in the proximal region, an opening between the outer tubular members adjacent to each other in the circumferential direction of the inner tubular member, the opening allowing a lumen of the inner tubular member to be in communication with a region outside the inner tubular member.
[8] The balloon catheter according to [4], in which the opening is located between the first outer tubular member and the second outer tubular member in the circumferential direction of the inner tubular member.
[6] The balloon catheter according to any one of [2] to [5], in which, in a cross-section in the proximal region and perpendicular to the longitudinal direction, a distance between an outer surface of the first outer tubular member and an outer surface of the second outer tubular member is larger than an outer diameter of each of (the outer tubular members constituting the tubular member group.
[7] The balloon catheter according to any one of [2] to [6], in which, in the proximal region, the tubular member group includes at least one set of the outer tubular members in which the outer tubular members adjacent to each other in the circumferential direction of the inner tubular member and constituting the tubular member group are in contact with each other, and at least one set of the outer tubular members in which the outer tubular members adjacent to each other in the circumferential direction of the inner tubular member and constituting the tubular member group are not in contact with each other.
[8] The balloon catheter according to [7], in which, among the outer tubular members constituting the tubular member group, the outer tubular members adjacent to each other in the circumferential direction of the inner tubular member are not in contact with each other in the distal region.
[9] The balloon catheter according to any one of [2] to [8], in which, in a cross-section in the distal region and perpendicular to the longitudinal direction, a distance between outer surfaces of the outer tubular members constituting the tubular member group, which are adjacent to each other in the circumferential direction of the inner tubular member, is smaller than an outer diameter of each of the outer tubular members.
[19] The balloon catheter according to any one of [2] to [9] , in which, in a cross-section in the distal region and perpendicular to the longitudinal direction, a distance between a center of the first outer tubular member and a center of the second outer tubular member is 90% or more and 110% or less of a distance between a center of the first outer tubular member and a center of the third outer tubular member.
[11] The balloon catheter according to any one of [1] to [10], in which the tubular member group includes the outer tubular members extending in a direction inclined with respect to a direction in which the inner tubular member extends.
[12] The balloon catheter according to [4], in which, on a proximal side with respect to the opening, the outer tubular members constituting the tubular member group are disposed to surround the inner tubular member in the circumferential direction of the inner tubular member.
[13] The balloon catheter according to [12],
in which the balloon catheter has an outer-tubular-member fixed region, which is a region in which the outer tubular members constituting the tubular member group are fixed to each other, and
in which the opening is arranged in the outer-tubular-member fixed region.
[14] The balloon catheter according to [4], further including:
a covering tube,
in which the balloon catheter has a member fixed region, which is a region in which the covering tube and each of the outer tubular members constituting the tubular member group are fixed to each other, and
in which the opening is arranged in the member fixed region.
[15] The balloon catheter according to [14],
in which, on a distal side with respect to the member fixed region, the covering tube is disposed with the inner tubular member and the tubular member group being disposed in a lumen of the covering tube, and
in which, on a proximal side with respect to the member fixed region, the covering tube is disposed without presence of the inner tubular member and the tubular member group.
[16] The balloon catheter according to [14],
in which, on a distal side with respect to the member fixed region, the inner tubular member and the tubular member group are disposed in a lumen of the covering tube, and
in which, on a proximal side with respect to the member fixed region, the tubular member group is disposed in the lumen of the covering tube without presence of the inner tubular member.
[17] The balloon catheter according to any one of [1] to [16],
in which a lumen of the inner tubular member is a guidewire lumen through which a guidewire is configured to be inserted, and
in which lumens of the outer tubular members constituting the tubular member group are inflation lumens through which a fluid to be supplied to lumens of the balloons is configured to pass, the inflation lumens each being in communication with a corresponding one of lumens of the plurality of balloons.
[18] The balloon catheter according to [17], further including:
a guidewire tube having a lumen in communication with the guidewire lumen,
in which the guidewire tube is disposed in a lumen of one of the balloons.

[19] The balloon catheter according to any one of [1] to [18], in which the balloon catheter is to be used for dilating an aortic valve, deforming a bioprosthetic valve placed in a heart, or destroying the bioprosthetic valve.

### Advantageous Effects of Invention

A method comprising dilating an aortic valve, deforming a bioprosthetic valve placed in a heart, or destroying the bioprosthetic valve, each being performed using the balloon catheter according to any one of [1] to [19].

According to the balloon catheter, in a cross-section perpendicular to the longitudinal direction, the shaft includes a portion where the distance between the center of the first outer tubular member and the center of the second outer tubular member is larger than the distance between the center of the first outer tubular member and the center of the third outer tubular member, and, consequently, a portion where the rigidity of the shaft is low due to a large gap between the first outer tubular member and the second outer tubular member in the circumferential direction of the inner tubular member and a portion where the rigidity of the shaft is high due to a small gap between the first outer tubular member and the third outer tubular member in the circumferential direction of the inner tubular member are provided adjacent to each other. As a result, the shaft has a structure in which the shaft is selectively easily bent between the first outer tubular member and the second outer tubular member, and it is possible to easily insert the shaft into a lumen in a living body by improving followability of the shaft.

### BRIEF DESCRIPTION OF DRAWINGS

FIG. 1 is a side view of a balloon catheter according to one or more embodiments of the present invention.

FIG. 2 is a side view of balloons of the balloon catheter illustrated in FIG. 1.

FIG. 3 is a side view of a shaft in a proximal region of the balloon catheter illustrated in FIG. 1.

FIG. 4 is a cross-sectional view of the shaft illustrated in FIG. 3 taken along the line IV-IV.

FIG. 5 is a side view of the shaft in a distal region of the balloon catheter illustrated in FIG. 1.

FIG. 6 is a cross-sectional view of the shaft illustrated in FIG. 5 taken along the line VI-VI.

FIG. 7 is a side view of a shaft in a proximal region of a balloon catheter according to one or more embodiments of the present invention.

FIG. 8 is a side view of a shaft in a proximal region of a balloon catheter according to one or more embodiments of the present invention.

### DETAILED DESCRIPTION

Hereinafter, one or more embodiments of the present invention will be described on the basis of embodiments, but the present invention is not limited to the following embodiments, and it is of course possible to carry out the present invention by adding modifications, as appropriate, within a range that can be adapted to the gist described above and below, and all of them are included in the technical scope of the present invention. In the drawings, hatching, reference signs of members, and the like may be omitted for convenience. In such a case, the description and other drawings are referred to. In addition, the dimensions of various members in the drawings may be different from actual dimensions thereof in order to facilitate understanding of the features of one or more embodiments of the present invention.

A balloon catheter according to one or more embodiments of the present invention includes a shaft that extends in the longitudinal direction from the proximal side toward the distal side and a plurality of balloons that are disposed at a distal portion of the shaft. The shaft includes an inner tubular member that has a lumen, and a tubular member group that is constituted by a plurality of outer tubular members that each have a lumen and that are disposed on the outer side with respect to the inner tubular member to be aligned in the circumferential direction of the inner tubular member. In a cross-section perpendicular to the longitudinal direction, the tubular member group includes a first outer tubular member, a second outer tubular member disposed adjacent to the first outer tubular member in the circumferential direction of the inner tubular member, and a third outer tubular member disposed adjacent to the first outer tubular member in the circumferential direction of the inner tubular member and on a side opposite to the second outer tubular member. In a cross-section perpendicular to the longitudinal direction, the shaft includes a portion in which a distance between the center of the first outer tubular member and the center of the second outer tubular member is larger than a distance between the center of the first outer tubular member and the center of the third outer tubular member.

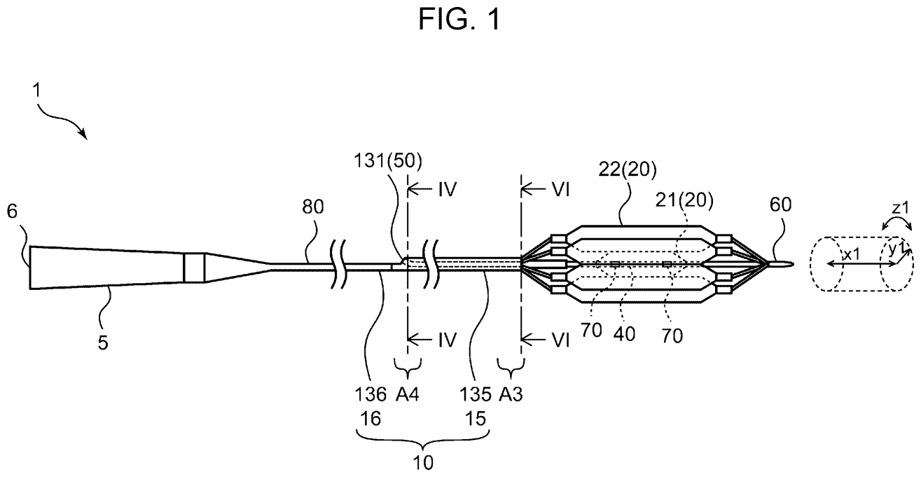

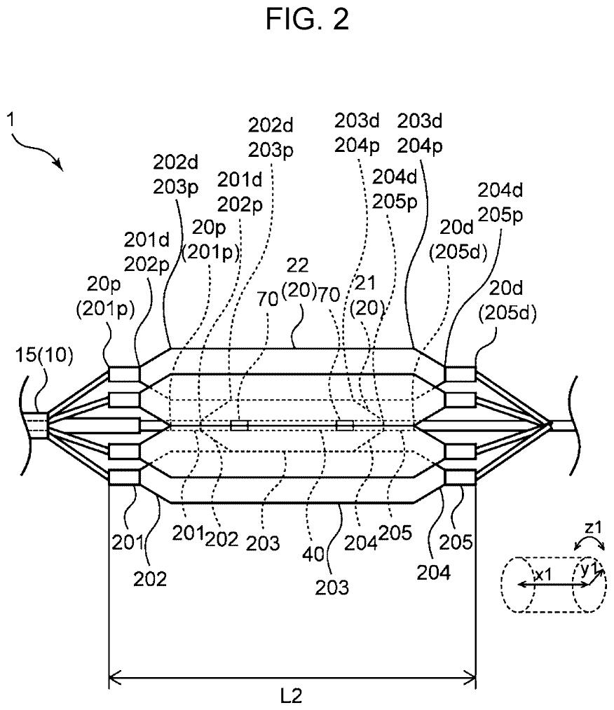

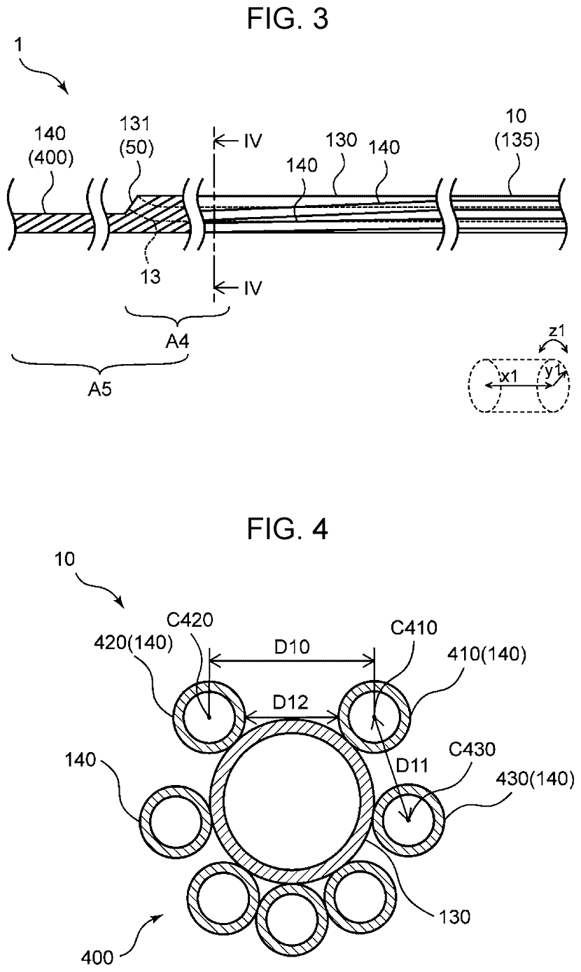

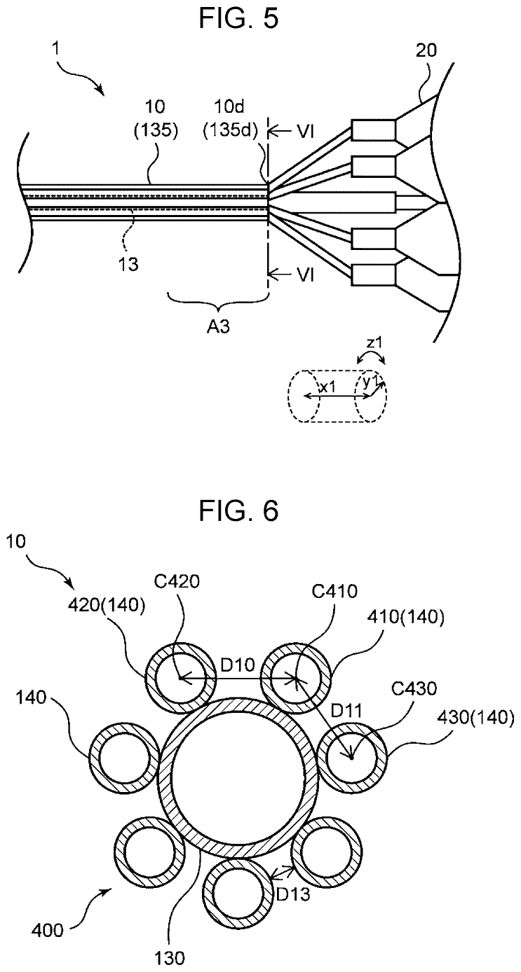

Hereinafter, a balloon catheter according to one or more embodiments of the present invention will be described with reference to FIG. 1 to FIG. 6. FIG. 1 is a side view of a balloon catheter according to one or more embodiments of the present invention, and FIG. 2 is a side view of balloons of the balloon catheter illustrated in FIG. 1. FIG. 3 is a side view of a shaft in a proximal region of the balloon catheter illustrated in FIG. 1, and FIG. 4 is a cross-sectional view of the shaft illustrated in FIG. 3 taken along the line IV-IV, the cross-sectional view indicating a cross-section perpendicular to the longitudinal direction of the shaft. FIG. 5 is a side view of the shaft in a distal region of the balloon catheter illustrated in FIG. 1, and FIG. 6 is a cross-sectional view of the shaft illustrated in FIG. 5 taken along the line VI-VI, the cross-sectional view indicating a cross-section perpendicular to the longitudinal direction of the shaft.

As illustrated in FIG. 1 and FIG. 2, a balloon catheter 1 includes a shaft 10 extending in a longitudinal direction from the proximal side toward the distal side, and a plurality of balloons 20 disposed at a distal portion of the shaft 10.

The shaft 10 has a longitudinal direction x1, a radial direction y1 connecting a centroid of an outer edge of the shaft 10 and a point on the outer edge in a cross-section perpendicular to the longitudinal direction x1, and a circumferential direction z1 along the outer edge of the shaft 10 in a cross-section perpendicular to the longitudinal direction x1. In the present description, in the longitudinal direction x1, a direction on the hand side of a user is referred to as the proximal side, and a side opposite to the proximal side, that is, a direction on the side of a treatment target is referred to as the distal side.

Members and portions other than the shaft 10 also each have a longitudinal direction, a radial direction, and a circumferential direction, which may be the same as or different from the longitudinal direction x1, the radial direction y1, and the circumferential direction z1 of the shaft 10, respectively. In the present description, for ease of understanding, all members and portions are described as having the same longitudinal direction, radial direction, and circumferential direction as the longitudinal direction x1, the radial direction y1, and the circumferential direction z1 of the shaft 10.

The balloons 20 are connected to a distal portion of the shaft 10. The balloons 20 can be inflated by introducing a fluid through a lumen of the shaft 10, and the balloons 20 can be deflated by discharging the fluid. In order to control the inflation and deflation of the balloons 20, a fluid can be introduced or discharged using an indeflator (balloon pressurizer). As the fluid, for example, physiological saline, a mixed solution of a contrast medium and physiological saline, or the like is used. The fluid may be a pressurized fluid that is pressurized by a pump or the like.

Examples of the material constituting the balloons 20 include polyamide resins such as nylon 11 and nylon 12, polyester resins such as polyethylene terephthalate and polybutylene terephthalate, polyurethane resins, and thermoplastic elastomers such as polyether block amide copolymers.

The materials constituting respective ones of the plurality of balloons 20 may be different from each other but may be the same. That is, the plurality of balloons 20 may be made of the same material. When the materials constituting respective ones of the plurality of balloons 20 are the same, it is possible to make the degree of inflation, hardness, and the like of the balloons 20 substantially the same in the circumferential direction z1 of the balloons 20.

In the inflated state of the balloons 20, respective maximum outer diameters of the plurality of balloons 20 may be different from each other but may be the same. The respective maximum outer diameters of the plurality of balloons 20 being the same means that the respective maximum outer diameters of the plurality of balloons 20 are substantially the same, and to be specific, means that the maximum outer diameter of one balloon 20 is 90% or more and 110% or less of each of the maximum outer diameters of all the other balloons 20. When the respective maximum outer diameters of the plurality of balloons 20 are the same in the inflated state of the balloons 20, the timing of inflation can be easily aligned in all the balloons 20, and the inflation of the balloons 20 can be easily controlled. The inflated state of the balloons 20 means a state in which a fluid is introduced into the lumens of all the balloons 20 and the balloons 20 are inflated.

As illustrated in FIG. 1 and FIG. 2, the plurality of balloons 20 may include a first balloon 21 and a plurality of second balloons 22 arranged on the outer side with respect to the first balloon 21 to be aligned in the circumferential direction of the first balloon 21. That is, the plurality of balloons 20 include the first balloon 21 and the plurality of second balloons 22, and the plurality of second balloons 22 may be disposed along the outer periphery of the first balloon 21.

The number of the first balloons 21 may be plural but may be one. That is, the balloon catheter 1 may have one first balloon 21 and a plurality of the second balloons 22. When the number of the first balloons 21 is one, the first balloon 21 is less likely to move on the inner side with respect to the plurality of second balloons 22 in the inflated state of the balloons 20. As a result, the inflation of the plurality of second balloons 22 is easily suppressed by the first balloon 21, and the hardness of the balloons 20 is increased, so that the inflation force can be easily increased.

The number of the second balloons 22 may be three or more, four or more, or five or more. By setting the lower limit value of the number of the second balloons 22 in the above range, the positions of the first balloon 21 and the second balloons 22 are less likely to be displaced in the inflated state of the balloons 20, and the inflation force of the balloons 20 can be increased. The number of the second balloons 22 may be 20 or less, 12 or less, 10 or less, or 8 or less. By setting the upper limit value of the number of the second balloons 22 in the above-described range, the outer diameter of a part including the balloons 20 is less likely to be excessively large, and the low invasiveness of the balloon catheter 1 can be improved.

In the inflated state of the plurality of balloons 20, the length from a distal end 20d of each balloon 20 to a proximal end 20p of the balloon 20 in the longitudinal direction x1 may be the same or different among the balloons 20. When the plurality of balloons 20 include the first balloon 21 and the plurality of second balloons 22, a length L2 from the distal end 20d of each second balloon 22 to the proximal end 20p of the second balloon 22 in the longitudinal direction x1 may be different but may be the same among the balloons 20. The fact that respective lengths L2 of the second balloons 22 from the distal end 20d to the proximal end 20p in the longitudinal direction x1 are the same means that the respective lengths L2 of the plurality of second balloons 22 in the longitudinal direction x1 are substantially the same, and to be specific, means that the length L2 of one second balloon 22 in the longitudinal direction x1 is 90% or more and 110% or less of each of the lengths L2 of all the other second balloons 22 in the longitudinal direction x1. When the respective lengths L2 of the plurality of second balloons 22 in the longitudinal direction x1 in the inflated state of the balloons 20 are the same, the timing of inflation of all the second balloons 22 can be easily aligned, and the inflation of the balloons 20 can be easily controlled.

As illustrated in FIG. 2, the balloons 20 may each include a straight tube portion 203, a proximal-side tapered portion 202 located on the proximal side with respect to the straight tube portion 203, a proximal-side sleeve portion 201 located on the proximal side with respect to the proximal-side tapered portion 202, a distal-side tapered portion 204 located on the distal side with respect to the straight tube portion 203, and a distal-side sleeve portion 205 located on the distal side with respect to the distal-side tapered portion 204.

The straight tube portion 203 may have a substantially cylindrical shape having substantially the same diameters in the longitudinal direction x1 but may have different diameters in the longitudinal direction x1. It is preferable that the proximal-side tapered portion 202 and the distal-side tapered portion 204 are each formed in a substantially conical shape or a truncated conical shape by being reduced in diameter with increasing distance from the straight tube portion 203. When the straight tube portion 203 has a maximum diameter, the straight tube portion 203 of each balloon 20 sufficiently comes into contact with a lesion portion when the balloons 20 are inflated in the lesion portion such as a stenosis portion, and thus it is possible to easily perform treatment such as dilation of the lesion portion. In addition, with the diameters of the proximal-side tapered portion 202 and the distal-side tapered portion 204 being reduced, when the balloons 20 are deflated, the outer diameters of a proximal end portion and a distal end portion of each of the balloons 20 can be reduced to reduce a step between the shaft 10 and each of the balloons 20. Therefore, the outer surface of the balloon catheter 1 becomes smooth in the deflated state of the balloons 20, and the balloon catheter 1 can be easily inserted into a body cavity.

In each balloon 20, while the proximal-side tapered portion 202, the straight tube portion 203, and the distal-side tapered portion 204 are portions that inflate when a fluid is introduced into the balloon 20, the proximal-side sleeve portion 201 and the distal-side sleeve portion 205 may be portions that do not inflate. When the proximal-side sleeve portion 201 and the distal-side sleeve portion 205 do not inflate, at least a part of the proximal-side sleeve portion 201 and at least a part of the distal-side sleeve portion 205 can be easily fixed to the shaft 10.

The shaft 10 has an inner tubular member 130 having a lumen, and a tubular member group 400 constituted by a plurality of outer tubular members 140 disposed on the outer side with respect to the inner tubular member 130 to be aligned in the circumferential direction of the inner tubular member 130 and each having a lumen. That is, the shaft 10 includes the inner tubular member 130 and the plurality of outer tubular members 140.

The inner tubular member 130 may be made of a resin, a metal, or a combination of a resin and a metal. By using a resin as the constituent material of the inner tubular member 130, it becomes easy to impart flexibility and elasticity to the inner tubular member 130. In addition, by using a metal as the constituent material of the inner tubular member 130, the deliverability of the balloon catheter 1 can be improved. Examples of the resin constituting the inner tubular member 130 include polyamide-based resin, polyester-based resin, polyurethane-based resin, polyolefin-based resin, fluorine-based resin, vinyl chloride-based resin, silicone-based resin, natural rubber, and synthetic rubbers. These may be used alone or in combination of two or more. The inner tubular member 130 is made of, for example, stainless steel such as SUS304 or SUS316, platinum, nickel, cobalt, chromium, titanium, tungsten, gold, Ni-Ti alloy, Co-Cr alloy, or a combination thereof. When the inner tubular member 130 is constituted by a distal-side inner tubular member 135 and a proximal-side inner tubular member 136 that are separate members, for example, the distal-side inner tubular member 135 may be formed of a resin, and the proximal-side inner tubular member 136 may be formed of a metal. Further, the inner tubular member 130 may have a laminated structure made of different materials or the same material.

The inner tubular member 130 has a lumen extending in the longitudinal direction x1. The number of lumens included in the inner tubular member 130 may be plural but may be one. When the number of lumens included in the inner tubular member 130 is one, the inner tubular member 130 is flexible, and it is possible to easily increase the flexibility of the shaft 10.

As materials constituting the outer tubular members 140, the materials exemplified as the materials constituting the inner tubular member 130 can be used. The materials constituting the outer tubular members 140 may be the same as or different from the material constituting the inner tubular member 130.

The outer tubular members 140 each have a lumen extending in the longitudinal direction x1. The number of lumens included in each of the outer tubular members 140 may be plural but may be one. When the number of lumens included in each of the outer tubular members 140 is one, the flexibility of the outer tubular members 140 is increased, and the shaft 10 having high flexibility is easily obtained.

The plurality of outer tubular members 140 constituting the tubular member group 400 may be made of different materials but may be made of the same material. That is, it is preferable that the tubular member group 400 is constituted by a plurality of the outer tubular members 140 made of the same material. When the plurality of outer tubular members 140 are made of the same material, it is possible to make the rigidity of the tubular member group 400 substantially the same in the circumferential direction z1 of the tubular member group 400.

Respective outer diameters of the plurality of outer tubular members 140 constituting the tubular member group 400 may be different from each other but may be the same. The fact that the outer diameters of the plurality of outer tubular members 140 constituting the tubular member group 400 are the same means that the outer diameters of the plurality of outer tubular members 140 constituting the tubular member group 400 are substantially the same, and to be specific, means that the outer diameter of one outer tubular member 140 is 90% or more and 110% or less of each of the outer diameters of all the other outer tubular members 140. When the respective outer diameters of the plurality of outer tubular members 140 constituting the tubular member group 400 are the same, the rigidity of all of the outer tubular members 140 constituting the tubular member group 400 becomes the same degree, and the flexibility of the shaft 10 in the circumferential direction z1 of the tubular member group 400 becomes likely to be the same degree.

The outer diameter of the inner tubular member 130 may be larger than the outer diameter of each of the outer tubular members 140. When the outer diameter of the inner tubular member 130 is larger than the outer diameter of each of the outer tubular members 140, the plurality of outer tubular members 140 are easily disposed on the outer side with respect to the inner tubular member 130 to be aligned in the circumferential direction z1, and the plurality of outer tubular members 140 are less likely to be displaced.

The outer diameter of the inner tubular member 130 may be more than or equal to 1.5 times, more than or equal to 2.0 times, or more than or equal to 2.5 times the outer diameter of each of the outer tubular members 140. By setting the lower limit value of the ratio between the outer diameter of the inner tubular member 130 and the outer diameter of each of the outer tubular members 140 within the above range, it is possible to easily dispose the plurality of outer tubular members 140 on the outer side with respect to the inner tubular member 130 to be aligned in the circumferential direction z1. In addition, the outer diameter of the inner tubular member 130 may be less than or equal to 10 times, less than or equal to 8 times, or less than or equal to 5 times the outer diameter of each of the outer tubular members 140. By setting the upper limit value of the ratio between the outer diameter of the inner tubular member 130 and the outer diameter of each of the outer tubular members 140 within the above range, the outer diameter of the shaft 10 is less likely to increase, and the shaft 10 can be easily made flexible.

The balloons 20 and the shaft 10 may be joined to each other by, for example, adhesion with an adhesive, welding, or crimping with a ring-shaped member attached to a portion where an end portion of each balloon 20 and the shaft 10 overlap each other. In particular, each balloon 20 and the shaft 10 may be joined to each other by welding. When each balloon 20 and the shaft 10 are joined to each other by welding, the joining between each balloon 20 and the shaft 10 is less likely to be released even when the balloons 20 are repeatedly inflated or deflated, and it is thus possible to improve the joining strength.

As illustrated in FIG. 1, a hub 5 may be provided on the proximal side of the shaft 10. In addition, the hub 5 may be provided with a fluid injection portion 6 in communication with a flow path of a fluid to be supplied to the inside of each balloon 20.

The shaft 10 and the hub 5 are joined to each other by, for example, adhesion with an adhesive, welding, or the like. In particular, the shaft 10 and the hub 5 may be joined to each other by adhesion. When the shaft 10 and the hub 5 are joined to each other by adhesion, the joining strength between the shaft 10 and the hub 5 can be increased to improve the durability of the balloon catheter 1 when the material of the shaft 10 is different from the material of the hub 5, for example, when the shaft 10 is made of a highly flexible material and the hub 5 is made of a highly rigid material.

As illustrated in FIG. 4 and FIG. 6, in a cross-section perpendicular to the longitudinal direction x1, the tubular member group 400 includes a first outer tubular member 410, a second outer tubular member 420 disposed adjacent to the first outer tubular member 410 in the circumferential direction z1 of the inner tubular member 130, and a third outer tubular member 430 disposed adjacent to the first outer tubular member 410 in the circumferential direction z1 of the inner tubular member 130 and on a side opposite to the second outer tubular member 420. That is, the tubular member group 400 includes the first outer tubular member 410, the second outer tubular member 420 disposed on one side in the circumferential direction z1 of the inner tubular member 130 with respect to the first outer tubular member 410, and the third outer tubular member 430 disposed on the other side in the circumferential direction z1 of the inner tubular member 130 with respect to the first outer tubular member 410. In other words, the second outer tubular member 420, the first outer tubular member 410, and the third outer tubular member 430 are arranged in this order toward one side in the circumferential direction z1 of the inner tubular member 130.

The first outer tubular member 410 and the second outer tubular member 420 are adjacent to each other in the circumferential direction z1 of the inner tubular member 130. Regarding the first outer tubular member 410 and the second outer tubular member 420, the first outer tubular member 410 and the second outer tubular member 420 may be in contact with each other, or a gap may be provided between the first outer tubular member 410 and the second outer tubular member 420.

The first outer tubular member 410 and the third outer tubular member 430 are adjacent to each other in the circumferential direction z1 of the inner tubular member 130. Regarding the first outer tubular member 410 and the third outer tubular member 430, the first outer tubular member 410 and the third outer tubular member 430 may be in contact with each other, or a gap may be provided between the first outer tubular member 410 and the third outer tubular member 430.

As illustrated in FIG. 3 and FIG. 4, in a cross-section perpendicular to the longitudinal direction x1, the shaft 10 includes a portion in which a distance D10 between a center C410 of the first outer tubular member 410 and a center C420 of the second outer tubular member 420 is larger than a distance D11 between the center C410 of the first outer tubular member 410 and a center C430 of the third outer tubular member 430. The center C410 of the first outer tubular member 410 in a cross-section perpendicular to the longitudinal direction x1 refers to the centroid of the outer edge of the cross-sectional shape of the first outer tubular member 410. Similarly, the center C420 of the second outer tubular member 420 in a cross-section perpendicular to the longitudinal direction x1 refers to the centroid of the outer edge of the cross-sectional shape of the second outer tubular member 420, and the center C430 of the third outer tubular member 430 in a cross-section perpendicular to the longitudinal direction x1 refers to the centroid of the outer edge of the cross-sectional shape of the third outer tubular member 430.

Since, in a cross-section perpendicular to the longitudinal direction x1, the shaft 10 includes a portion in which the distance D10 between the center C410 of the first outer tubular member 410 and the center C420 of the second outer tubular member 420 is larger than the distance D11 between the center C410 of the first outer tubular member 410 and the center C430 of the third outer tubular member 430, the distance between the first outer tubular member 410 and the second outer tubular member 420 in the circumferential direction z1 of the inner tubular member 130 can be made larger, at at least a portion of the shaft 10, than the distance between the first outer tubular member 410 and the third outer tubular member 430 in the circumferential direction z1 of the inner tubular member 130. The shaft 10 has, in a cross-section perpendicular to the longitudinal direction x1, a portion where the rigidity of the shaft 10 is low due to a large gap between the first outer tubular member 410 and the second outer tubular member 420 in the circumferential direction z1 of the inner tubular member 130 and a portion where the rigidity of the shaft 10 is high due to a small gap between the first outer tubular member 410 and the third outer tubular member 430 in the circumferential direction z1 of the inner tubular member 130 such that the portions are adjacent to each other. As a result, the shaft 10 has a structure that is selectively easily bendable between the first outer tubular member 410 and the second outer tubular member 420. Since the shaft 10 has a selectively easily bendable structure, it is possible to improve the followability of the shaft 10, for example, in a blood vessel having a large bend such as an aortic arch.

In a cross-section perpendicular to the longitudinal direction x1, the distance D10 between the center C410 of the first outer tubular member 410 and the center C420 the second outer tubular member 420 may be more than or equal to 1.5 times, more than or equal to 1.7 times, or more than or equal to 2.0 times the distance D11 between the center C410 of the first outer tubular member 410 and the center C430 of the third outer tubular member 430. By setting the lower limit value of the ratio between the distance D10 between the center C410 of the first outer tubular member 410 and the center C420 of the second outer tubular member 420 and the distance D11 between the center C410 of the first outer tubular member 410 and the center C430 of the third outer tubular member 430 within the above range, the gap between the first outer tubular member 410 and the second outer tubular member 420 can be easily increased in the circumferential direction z1 of the inner tubular member 130, and the rigidity of the shaft 10 between the first outer tubular member 410 and the second outer tubular member 420 can be reduced. In a cross-section perpendicular to the longitudinal direction x1, the distance D10 between the center C410 of the first outer tubular member 410 and the center C420 of the second outer tubular member 420 may be less than or equal to 10 times, less than or equal to 7 times, or less than or equal to 5 times the distance D11 between the center C410 of the first outer tubular member 410 and the center C430 of the third outer tubular member 430. By setting the upper limit value of the ratio between the distance D10 between the center C410 of the first outer tubular member 410 and the center C420 of the second outer tubular member 420 and the distance D11 between the center C410 of the first outer tubular member 410 and the center C430 of the third outer tubular member 430 within the above range, there is provided a portion in which a gap between the first outer tubular member 410 and the third outer tubular member 430 in the circumferential direction z1 of the inner tubular member 130 is small and in which rigidity is high while the distance between the first outer tubular member 410 and the second outer tubular member 420 is maintained in the circumferential direction z1 of the inner tubular member 130. Therefore, it is possible to easily achieve a structure in which the shaft 10 is selectively easily bent between the first outer tubular member 410 and the second outer tubular member 420.

As illustrated in FIG. 1, FIG. 3, and FIG. 5, the shaft 10 may have a distal region A3, which is a region on the distal side, and a proximal region A4, which is a region located on the proximal side with respect to the distal region A3.

The distal region A3 includes the distal end of the inner tubular member 130 and may be a region that extends from the distal end of the inner tubular member 130 toward the proximal side by 1/30 or more of the length in the longitudinal direction x1 from the distal end of the inner tubular member 130 to the proximal end of the inner tubular member 130, a region that extends from the distal end of the inner tubular member 130 toward the proximal side by 1/20 or more of the length in the longitudinal direction x1 from the distal end of the inner tubular member 130 to the proximal end of the inner tubular member 130, or a region that extends from the distal end of the inner tubular member 130 toward the proximal side by 1/10 or more of the length in the longitudinal direction x1 from the distal end of the inner tubular member 130 to the proximal end of the inner tubular member 130.

The proximal region A4 is located on the proximal side with respect to the distal region A3 and may be a region of 1/50 or more of the length in the longitudinal direction x1 from the distal end of the inner tubular member 130 to the proximal end of the inner tubular member 130, a region of 1/30 or more of the length in the longitudinal direction x1 from the distal end of the inner tubular member 130 to the proximal end of the inner tubular member 130, or a region of 1/20 or more of the length in the longitudinal direction x1 from the distal end of the inner tubular member 130 to the proximal end of the inner tubular member 130.

When the inner tubular member 130 is configured to have the distal-side inner tubular member 135 and the proximal-side inner tubular member 136, the distal region A3 includes a distal end 135d of the distal-side inner tubular member 135 and may be a region that extends from the distal end 135d of the distal-side inner tubular member 135 toward the proximal side by 1/30 or more of the length in the longitudinal direction x1 from the distal end 135d of the distal-side inner tubular member 135 to the proximal end of the distal-side inner tubular member 135, a region that extends from the distal end 135d of the distal-side inner tubular member 135 toward the proximal side by 1/20 or more of the length in the longitudinal direction x1 from the distal end 135d of the distal-side inner tubular member 135 to the proximal end of the distal-side inner tubular member 135, or a region that extends from the distal end 135d of the distal-side inner tubular member 135 toward the proximal side by 1/10 or more of the length in the longitudinal direction x1 from the distal end 135d of the distal-side inner tubular member 135 to the proximal end of the distal-side inner tubular member 135. In addition, the proximal region A4 is located on the proximal side with respect to the distal region A3 and includes the proximal end of the distal-side inner tubular member 135, and the proximal region A4 may be a region extending from the proximal end of the distal-side inner tubular member 135 toward the distal side by 1/50 or more of the length from the distal end 135d of the distal-side inner tubular member 135 to the proximal end of the distal-side inner tubular member 135 in the longitudinal direction x1, a region extending from the proximal end of the distal-side inner tubular member 135 toward the distal side by 1/30 or more of the length from the distal end 135d of the distal-side inner tubular member 135 to the proximal end of the distal-side inner tubular member 135 in the longitudinal direction x1, or a region extending from the proximal end of the distal-side inner tubular member 135 toward the distal side by 1/20 or more of the length from the distal end 135d of the distal-side inner tubular member 135 to the proximal end of the distal-side inner tubular member 135 in the longitudinal direction x1.

As illustrated in FIG. 4, in a cross-section in the proximal region A4 and perpendicular to the longitudinal direction x1, the distance D10 between the center C410 of the first outer tubular member 410 and the center C420 of the second outer tubular member 420 may be larger than the distance D11 between the center C410 of the first outer tubular member 410 and the center C430 of the third outer tubular member 430.

In cross-section in the proximal region A4 and perpendicular to the longitudinal direction x1, when the distance D10 between the center C410 of the first outer tubular member 410 and the center C420 of the second outer tubular member 420 is larger than the distance D11 between the center C410 of the first outer tubular member 410 and the center C430 of the third outer tubular member 430, the distance between the first outer tubular member 410 and the second outer tubular member 420 in the circumferential direction z1 of the inner tubular member 130 can be larger than the distance between the first outer tubular member 410 and the third outer tubular member 430 in the circumferential direction z1 of the inner tubular member 130. In a cross-section in the proximal region A4, which is located away from the tip of the shaft 10 by a certain distance, and perpendicular to the longitudinal direction x1, a portion where the rigidity of the shaft 10 is low due to the large gap between the first outer tubular member 410 and the second outer tubular member 420 in the circumferential direction z1 of the inner tubular member 130 and a portion where the rigidity of the shaft 10 is high due to the small gap between the first outer tubular member 410 and the third outer tubular member 430 in the circumferential direction z1 of the inner tubular member 130 are provided adjacent to each other. As a result, the shaft 10 has a structure that is selectively easily bendable between the first outer tubular member 410 and the second outer tubular member 420. Since the shaft 10 has a selectively easily bendable structure, for example, it is possible to improve the followability of the shaft 10 in a blood vessel having a large bend such as an aortic arch.

In the longitudinal direction x1, the length of the proximal region A4 may be less than or equal to the length of the distal region A3. When the length of the proximal region A4 is the same as the length of the distal region A3 or shorter than the length of the distal region A3, the length of the proximal region A4 having high flexibility and the length of the distal region A3 having appropriate rigidity are balanced in the entire shaft 10, and the shaft 10 having favorable insertability can be obtained. The length of the proximal region A4 may be shorter than the length of the distal region A3 in the longitudinal direction x1. When the length of the proximal region A4 is shorter than the length of the distal region A3, the insertability of the shaft 10 can be further improved.

As illustrated in FIG. 1 and FIG. 3, the inner tubular member 130 may have, in the proximal region A4 and between the outer tubular members 140 adjacent to each other in the circumferential direction z1 of the inner tubular member 130, an opening 131 through which the lumen of the inner tubular member 130 is in communication with a region outside the inner tubular member 130. When the inner tubular member 130 has the opening 131 in the proximal region A4 and between the adjacent outer tubular members 140, the balloon catheter 1 is of a so-called rapid exchange type, and an article such as a guidewire, a fluid, or the like can be easily introduced into the lumen of the inner tubular member 130 through the opening 131. As a result, time of manipulation using the balloon catheter 1 can be shortened, and the low invasiveness can be improved.

The opening 131 may be positioned between the first outer tubular member 410 and the second outer tubular member 420 in the circumferential direction z1 of the inner tubular member 130. When the opening 131 is positioned between the first outer tubular member 410 and the second outer tubular member 420, the opening 131 is positioned between the first outer tubular member 410 and the second outer tubular member 420, which are spaced apart from each other in the circumferential direction z1 of the inner tubular member 130. Therefore, when an article such as a guidewire is inserted through the opening 131, the article inserted through the opening 131 is less likely to come into contact with the outer tubular members 140, and the outer tubular members 140 can be less likely to be damaged.

As illustrated in FIG. 4, in a cross-section in the proximal region A4 and perpendicular to the longitudinal direction x1, a distance D12 between the outer surface of the first outer tubular member 410 and the outer surface of the second outer tubular member 420 may be larger than the outer diameter of each of the outer tubular members 140 constituting the tubular member group 400. The distance D12 between the outer surfaces of the first outer tubular member 410 and the second outer tubular member 420 is the length of the shortest one of straight lines each connecting a point on the outer edge of the first outer tubular member 410 to a point on the outer edge of the second outer tubular member 420. That is, in a cross-section in the proximal region A4 and perpendicular to the longitudinal direction x1, the interval between the first outer tubular member 410 and the second outer tubular member 420 may be larger than the outer diameter of each of the outer tubular members 140. When the distance D12 between the outer surfaces of the first and second outer tubular members 410 and 420 is larger than the outer diameter of each of the outer tubular members 140 constituting the tubular member group 400, the interval between the first and second outer tubular members 410 and 420 can be increased, and the rigidity of the shaft 10 in the proximal region A4 can be increased to improve the insertability of the shaft 10.

In a cross-section in the proximal region A4 and perpendicular to the longitudinal direction x1, the distance D12 between the outer surfaces of the first outer tubular member 410 and the second outer tubular member 420 may be more than or equal to 2.0 times, more than or equal to 2.5 times, or more than or equal to 3.0 times the outer diameter of each of the outer tubular members 140 constituting the tubular member group 400. By setting the lower limit value of the ratio between the distance D12 between the outer surfaces of the first and second outer tubular members 410 and 420 and the outer diameter of each of the outer tubular members 140 constituting the tubular member group 400 within the above range, a sufficient gap can be secured between the first and second outer tubular members 410 and 420, and the rigidity of the shaft 10 in the proximal region A4 can be easily increased. In a cross-section in the proximal region A4 and perpendicular to the longitudinal direction x1, the distance D12 between the outer surfaces of the first outer tubular member 410 and the second outer tubular member 420 may be less than or equal to 10 times, less than or equal to 7 times, or less than or equal to 5 times the outer diameter of each of the outer tubular members 140 constituting the tubular member group 400. By setting the upper limit value of the ratio between the distance D12 between the outer surfaces of the first outer tubular member 410 and the second outer tubular member 420 and the outer diameter of each of the outer tubular members 140 constituting the tubular member group 400 within the above-described range, the outer diameter of the shaft 10 is unlikely to be excessively large, and the shaft 10 is likely to be made minimally invasive.

As illustrated in FIG. 4, in the proximal region A4, the tubular member group 400 may include at least one set of outer tubular members 140 in which the outer tubular members 140 adjacent to each other in the circumferential direction z1 of the inner tubular member 130 and constituting the tubular member group 400 are in contact with each other, and at least one set of outer tubular members 140 in which the outer tubular members 140 adjacent to each other in the circumferential direction z1 of the inner tubular member 130 and constituting the tubular member group 400 are not in contact with each other. That is, the tubular member group 400 may include, in the proximal region A4, both the outer tubular members 140 in contact with the outer tubular members 140 adjacent thereto in the circumferential direction z1 of the inner tubular member 130 and the outer tubular members 140 in contact with the outer tubular members 140 adjacent thereto in the circumferential direction z1 of the inner tubular member 130. In the proximal region A4, due to the tubular member group 400 including at least one set of outer tubular members 140 in which the outer tubular members 140 adjacent to each other in the circumferential direction z1 of the inner tubular member 130 and constituting the tubular member group 400 are in contact with each other and at least one set of outer tubular members 140 in which the outer tubular members 140 adjacent to each other in the circumferential direction z1 of the inner tubular member 130 and constituting the tubular member group 400 are not in contact with each other, it is possible to increase rigidity of the shaft 10 in the proximal region A4 so that a force applied from the proximal side is easily transmitted toward the distal side and also possible to impart a certain degree of flexibility to the shaft 10.

As illustrated in FIG. 5 and FIG. 6, in the distal region A3, the outer tubular members 140 constituting the tubular member group 400 may be arranged such that the outer tubular members 140 adjacent to each other in the circumferential direction z1 of the inner tubular member 130 are not in contact with each other. When the outer tubular members 140 adjacent to each other are not in contact with each other in the distal region A3, the flexibility of the shaft 10 in the distal region A3 can be increased, and the shaft 10 can easily follow a lumen in a living body such as a blood vessel.

As illustrated in FIG. 6, in a cross-section in the distal region A3 and perpendicular to the longitudinal direction x1, the distance D13 between the outer surfaces of the outer tubular members 140 adjacent to each other in the circumferential direction z1 of the inner tubular member 130 constituting the tubular member group 400 may be smaller than the outer diameter of each of the outer tubular members 140. The distance D13 between the outer surfaces of the outer tubular members 140 refers to the length of the shortest one of straight lines each connecting a point on the outer edge of one of the outer tubular members 140 adjacent to each other in the circumferential direction z1 of the inner tubular member 130 to a point on the outer edge of the other one of the outer tubular members 140. That is, in a cross-section in the distal region A3 and perpendicular to the longitudinal direction x1, the interval between the adjacent outer tubular members 140 may be larger than the outer diameter of each of the outer tubular members 140. When the distance D13 between the outer surfaces of the adjacent outer tubular members 140 is smaller than the outer diameter of each of the outer tubular members 140, it is possible to impart appropriate rigidity to the shaft 10 in the distal region A3, and it is possible to make the shaft 10 have favorable insertability by improving pushability.

In a cross-section in the distal region A3 and perpendicular to the longitudinal direction x1, the distance D13 between the outer surfaces of the outer tubular members 140 adjacent to each other in the circumferential direction z1 of the inner tubular member 130 and constituting the tubular member group 400 may be 80% or less, 70% or less, or 60% or less of the outer diameter of each of the outer tubular members 140. By setting the upper limit value of the ratio between the distance D13 between the outer surfaces of the outer tubular members 140 adjacent to each other and the outer diameter of each of the outer tubular members 140 within the above-described range, the interval between the outer tubular members 140 adjacent to each other can be narrowed, and the rigidity of the shaft 10 in the distal region A3 can be appropriately increased. In addition, in a cross-section in the distal region A3 and perpendicular to the longitudinal direction x1, the distance D13 between the outer surfaces of the outer tubular members 140 adjacent to each other in the circumferential direction z1 of the inner tubular member 130 and constituting the tubular member group 400 may be 10% or more, 15% or more, or 20% or more of the outer diameter of each of the outer tubular members 140. By setting the lower limit value of the ratio between the distance D13 between the outer surfaces of the adjacent outer tubular members 140 and the outer diameter of each of the outer tubular members 140 within the above-described range, the flexibility of the shaft 10 in the distal region A3 can be improved, and the shaft 10 can have favorable flexibility.

As illustrated in FIG. 6, in a cross-section in the distal region A3 and perpendicular to the longitudinal direction x1, the distance D10 between the center C410 of the first outer tubular member 410 and the center C420 of the second outer tubular member 420 may be 90% or more and 110% or less of the distance D11 between the center C410 of the first outer tubular member 410 and the center C430 of the third outer tubular member 430. By setting the ratio between the distance D10 between the center C410 of the first outer tubular member 410 and the center C420 of the second outer tubular member 420 and the distance D11 between the center C410 of the first outer tubular member 410 and the center C430 of the third outer tubular member 430 within the above range in a cross-section in the distal region A3 and perpendicular to the longitudinal direction x1, the intervals at which the plurality of outer tubular members 140 are arranged are substantially the same in the distal region A3. That is, in the proximal region A4, a plurality of the outer tubular members 140 are arranged unevenly in the circumferential direction z1 of the inner tubular member 130, 1 whereas in the distal region A3, a plurality of the outer tubular members 140 are arranged relatively evenly in the circumferential direction z1 of the inner tubular member 130. As a result, a force applied from the hand side of the shaft 10 is easily transmitted uniformly, and the insertability of the shaft 10 can be improved.

In a cross-section in the distal region A3 and perpendicular to the longitudinal direction x1, the distance D10 between the center C410 of the first outer tubular member 410 and the center C420 of the second outer tubular member 420 may be 90% or more, 92% or more, or 95% or more of the distance D11 between the center C410 of the first outer tubular member 410 and the center C430 of the third outer tubular member 430, and may be 110% or less, 108% or less, or 105% or less of the distance D11. In a cross-section in the distal region A3 and perpendicular to the longitudinal direction x1, by setting the ratio between the distance D10 between the center C410 of the first outer tubular member 410 and the center C420 of the second outer tubular member 420 and the distance D11 between the center C410 of the first outer tubular member 410 and the center C430 of the third outer tubular member 430 within the above range, the intervals at which the plurality of outer tubular members 140 are disposed in the distal region A3 are likely to be substantially the same, and the insertability of the shaft 10 can be further improved.

As illustrated in FIG. 3, the tubular member group 400 may include the outer tubular members 140 extending in a direction inclined with respect to the extending direction of the inner tubular member 130. In other words, the tubular member group 400 may include the outer tubular member 140 extending in a direction that is inclined with respect to a direction in which the inner tubular member 130 extends. When the tubular member group 400 includes the outer tubular member 140 extending in a direction inclined with respect to the extending direction of the inner tubular member 130, there are the outer tubular members 140 extending in directions different from the extending direction of the inner tubular member 130. As a result, the arrangement of the tubular member group 400 constituted by the plurality of outer tubular members 140 in a cross-section perpendicular to the longitudinal direction x1 varies in the longitudinal direction x1, and it is possible to make the characteristics of the shaft 10 different between the distal side and the proximal side, such as a configuration in which the flexibility of the shaft 10 increases on the distal side of the shaft 10 and the rigidity of the shaft 10 increases on the proximal side of the shaft 10.

On the proximal side with respect to the opening 131, the outer tubular members 140 constituting the tubular member group 400 may be disposed to surround the inner tubular member 130 in the circumferential direction z1 of the inner tubular member 130. When the outer tubular members 140 are disposed on the proximal side with respect to the opening 131 to surround the inner tubular member 130 in the circumferential direction z1 of the inner tubular member 130, a portion of the shaft 10 on the proximal side with respect to the opening 131 is supported by the outer tubular members 140, the rigidity of the proximal side of the shaft 10 is increased, and thus the pushability of the shaft 10 can be increased.

On the proximal side with respect to the opening 131, the outer tubular members 140 constituting the tubular member group 400 may be disposed to spirally surround the inner tubular member 130 in the circumferential direction z1 of the inner tubular member 130. When the outer tubular members 140 are disposed on the proximal side with respect to the opening 131 to spirally surround the inner tubular member 130 in the circumferential direction z1 of the inner tubular member 130, it is possible to improve pushability of the shaft 10 while imparting flexibility to the portion of the shaft 10 on the proximal side with respect to the opening 131 to make the shaft 10 easy to bend.

As illustrated in FIG. 3, the balloon catheter 1 has an outer-tubular-member fixed region A5, which is a region where the outer tubular members 140 constituting the tubular member group 400 are fixed to each other, and the opening 131 may be arranged in the outer-tubular-member fixed region A5. When the opening 131 is arranged in the outer-tubular-member fixed region A5, the rigidity of the portion of the inner tubular member 130 where the opening 131 is provided can be further increased, and the rigidity of the proximal side of the shaft 10 can be increased to improve the insertability of the shaft 10.

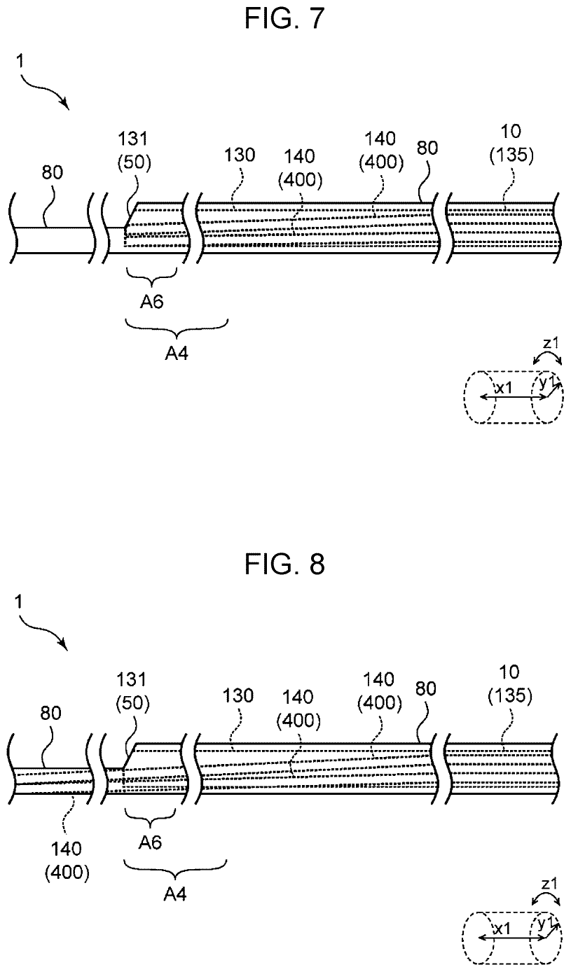

FIG. 7 is a side view of a shaft in a proximal region of a balloon catheter according to one or more embodiments of the present invention, and FIG. 8 is a side view of a shaft in a proximal region of a balloon catheter according to one or more embodiments of the present invention.

As illustrated in FIG. 7 and FIG. 8, it is also preferable that the balloon catheter 1 further includes a covering tube 80 and has a member fixed region A6 in which the covering tube 80 and each of the outer tubular members 140 constituting the tubular member group 400 are fixed to each other and that the opening 131 is arranged in the member fixed region A6. When the covering tube 80 and each of the outer tubular members 140 constituting the tubular member group 400 are fixed to each other in the member fixed region A6, the plurality of outer tubular members 140 are easily fixed by the covering tube 80 and are not easily displaced. In addition, when the opening 131 is arranged in the member fixed region A6, it is possible to increase the rigidity of the portion in which the opening 131 is provided, and it is possible to obtain the shaft 10 having favorable insertability.

Examples of the material constituting the covering tube 80 include polyamide-based resin, polyester-based resin, polyurethane-based resin, polyolefin-based resin, fluorine-based resin, vinyl chloride-based resin, silicone-based resin, natural rubber, and synthetic rubbers. These may be used alone or in combination of two or more. In particular, the material constituting the covering tube 80 may contain the same resin as a resin constituting the shaft 10. When the material constituting the covering tube 80 contains the same resin as the resin constituting the shaft 10, the joining strength between the covering tube 80 and the shaft 10 is easily increased.

The covering tube 80 and the shaft 10 may be joined to each other by, for example, adhesion with an adhesive or welding. In particular, the covering tube 80 and the shaft 10 may be joined to each other by welding. When the covering tube 80 and the shaft 10 are joined to each other by welding, the joining between the covering tube 80 and the shaft 10 is not easily released, and the joining strength can be improved.

As illustrated in FIG. 7, it is preferable that, on the distal side with respect to the member fixed region A6, the covering tube 80 is disposed with the inner tubular member 130 and the tubular member group 400 being disposed in the lumen of the covering tube 80, and, on the proximal side with respect to the member fixed region A6, the covering tube 80 is disposed without presence of the inner tubular member 130 and the tubular member group 400. When the inner tubular member 130 and the tubular member group 400 are disposed in the lumen of the covering tube 80 on the distal side with respect to the member fixed region A6 and when the inner tubular member 130 and the tubular member group 400 are not present on the proximal side with respect to the member fixed region A6, the outer diameter of the shaft 10 can be reduced in the portion on the proximal side with respect to the member fixed region A6, and the shaft 10 can have favorable passability.

Further, as illustrated in FIG. 8, it is also preferable that, on the distal side with respect to the member fixed region A6, the inner tubular member 130 and the tubular member group 400 are disposed in the lumen of the covering tube 80 and, on the proximal side with respect to the member fixed region A6, the tubular member group 400 is disposed in the lumen of the covering tube 80 without presence of the inner tubular member 130. When, on the proximal side with respect to the member fixed region A6, the tubular member group 400 is disposed in the lumen of the covering tube 80 without presence of the inner tubular member 130, the tubular member group 400 is present in the lumen of the covering tube 80 on the proximal side with respect to the member fixed region A6, the rigidity is increased in the portion on the proximal side with respect to the member fixed region A6, and the shaft 10 having favorable insertability can be obtained.

As illustrated in FIG. 3 and FIG. 5, the lumen of the inner tubular member 130 may be a guidewire lumen 13 through which a guidewire is to be inserted, the lumens of the outer tubular members 140 constituting the tubular member group 400 are inflation lumens through which a fluid to be supplied to the lumens of the balloons 20 passes, and the lumens of the outer tubular members 140 each communicate with a corresponding one of the lumens of the plurality of balloons 20. When the lumen of the inner tubular member 130 is the guidewire lumen 13 and the lumens of the outer tubular members 140 are the inflation lumens, it is possible to easily insert a guidewire into the inner tubular member 130 having the guidewire lumen 13 and to make the outer tubular members 140 less likely to be damaged when the guidewire is inserted into the inner tubular member 130.

As illustrated in FIG. 1 and FIG. 2, a guidewire tube 40 having a lumen communicating with the guidewire lumen 13 may be further included, and the guidewire tube 40 may be disposed in the lumen of one of the balloons 20. When the balloon catheter 1 includes the guidewire tube 40 having the lumen communicating with the guidewire lumen 13, a guidewire can be easily inserted into the balloon catheter 1, and the balloon catheter 1 can be conveyed along the guidewire into a body. Further, by inserting a guidewire into the guidewire tube 40, it is possible to prevent the guidewire from damaging the balloons 20 and the like.

Examples of the material constituting the guidewire tube 40 include synthetic resins and the like including polyolefin-based resin such as polyethylene and polypropylene; polyamide-based resin such as nylon; polyester-based resin such as PET; aromatic polyether ketone-based resin such as PEEK; polyether polyamide-based resin; polyurethane-based resin; polyimide-based resin; fluorine-based resin such as PTFE, PFA, and ETFE; and polyvinyl chloride-based resin. In particular, the material constituting the guidewire tube 40 may be the polyimide-based resin. When the material constituting the guidewire tube 40 is the polyimide-based resin, the slidability of the guidewire tube 40 is improved. Therefore, it is easy to insert a guidewire into the lumen of the guidewire tube 40 and to advance the balloon catheter 1 into a body along the guidewire. Further, the guidewire tube 40 may have a multi-layer structure including a braid layer such as a metal braid. When the guidewire tube 40 has a multilayer structure, the strength, the slidability with respect to a guidewire, and the kink resistance of the guidewire tube 40 can be enhanced.

The opening 131 may be a guidewire port 50 for inserting a guidewire into the lumen of the inner tubular member 130. That is, the balloon catheter 1 of one or more embodiments of the present invention may be a so-called rapid-exchange-type balloon catheter 1. The balloon catheter 1 may include a distal-side shaft 15 and a proximal-side shaft 16. The distal-side shaft 15 and the proximal-side shaft 16 may be separate members, and a proximal end portion of the distal-side shaft 15 may be connected to a distal end portion of the proximal-side shaft 16 to constitute the shaft 10 extending from the balloons 20 to a proximal end portion of the balloon catheter 1. Alternatively, one shaft 10 may extend from the balloons 20 to the proximal end portion of the balloon catheter 1, and the distal-side shaft 15 and the proximal-side shaft 16 may be further constituted by a plurality of tube members.

As illustrated in FIG. 1, a proximal end portion of the guidewire tube 40 may be connected to a distal end portion of the shaft 10. When the shaft 10 is configured to include the distal-side shaft 15 and the proximal-side shaft 16, a proximal end portion of the guidewire tube 40 may be connected to a distal end portion of the distal-side shaft 15. When the proximal end portion of the guidewire tube 40 is connected to the distal end portion of the shaft 10, the outer diameter of the balloon catheter 1 is less likely to be large, and the low invasiveness can be improved.

The distal end portion of the balloon catheter 1 may be provided with an end tip member 60. The end tip member 60 may be provided, as a separate member from the guidewire tube 40, at a distal end portion of the balloon catheter 1 by being connected to distal end portions of the balloons 20, or the guidewire tube 40 extending to the distal side with respect to the distal ends 20d of the balloons 20 may function as the end tip member 60.

As illustrated in FIG. 1 and FIG. 2, radiopaque markers 70 may be disposed on the guidewire tube 40 inside the balloon 20 to be located at portions where the balloons 20 are located in the longitudinal direction xl so that the positions of the balloons 20 can be confirmed by X-ray fluoroscopy.

Examples of the positions on the guidewire tube 40 at which the radiopaque markers 0 are disposed include the position of a midpoint of a length from the distal ends 20d of the balloons 20 to the proximal ends 20p of the balloons 20, and the positions of a proximal end 203p and a distal end 203d of the straight tube portion 203 of each of the balloons 20. In particular, the positions on the guidewire tube 40 at which the radiopaque markers 70 are disposed may be the positions of the proximal end 203p and the distal end 203d of the straight tube portion 203 of each balloon 20. When the radiopaque markers 70 are disposed, on the guidewire tube 40, at positions located at the proximal end 203p and the distal end 203d of the straight tube portion 203 of each balloon 20, the positions of the balloons 20 can be easily confirmed. As a result, it is possible to provide the balloon catheter 1 capable of easily applying pressure to a target site.

In the shaft 10, the outer wall of at least one of the distal-side shaft 15 and the proximal-side shaft 16 may be coated, and it is more preferable that the outer walls of both the distal-side shaft 15 and the proximal-side shaft 16 are coated.

The coating applied to the shaft 10 can be a hydrophilic coating or a hydrophobic coating depending on the purpose, and the coating can be applied by immersing the shaft 10 in a hydrophilic coating agent or a hydrophobic coating agent, applying a hydrophilic coating agent or a hydrophobic coating agent to the outer wall of the shaft 10, or coating the outer wall of the shaft 10 with a hydrophilic coating agent or a hydrophobic coating agent. The coating agent may contain chemicals, additives, and the like.

Examples of the hydrophilic coating agent include hydrophilic coating agents made of hydrophilic polymers such as polyvinyl alcohol, polyethylene glycol, polyacrylamide, polyvinylpyrrolidone, and methyl vinyl ether-maleic anhydride copolymer, or any combination thereof.

Examples of the hydrophobic coating agent include polytetrafluoroethylene (PTFE) , fluorinated ethylene propylene (FEP), perfluoroalkoxyalkane (PFA), silicone oil, hydrophobic urethane resin, carbon coating, diamond coating, diamond-like carbon (DLC) coating, ceramic coating, and a substance terminated with an alkyl group or a perfluoroalkyl group and having low surface free energy.

The balloon catheter 1 of one or more embodiments of the present invention may be used for dilation of an aortic valve, deformation of a bioprosthetic valve placed in a heart, or destruction of a bioprosthetic valve. Specifically, it is preferable that the balloon catheter 1 of one or more embodiments of the present invention is used for the purpose of dilating an aortic valve hardened by calcification or the like, or deforming or destroying an artificial valve annulus or the like of a bioprosthetic valve to replace a bioprosthetic valve placed in a heart and deteriorated. The balloon catheter 1 of one or more embodiments of the present invention can have a structure having a plurality of the balloons 20 because the partition walls between the plurality of lumens of the shaft 10 are less likely to be damaged and a fluid or an article fed into one lumen is less likely to enter other lumens, and can be suitably used because dilation of a hardened aortic valve or deformation or destruction of a bioprosthetic valve, which cannot be sufficiently performed by a conventional balloon catheter, can be easily performed.

The present application claims the benefit of priority based on Japanese Patent Application No. 2023-102818 filed on Jun. 22, 2023. The entire disclosure of Japanese Patent Application No. 2023-102818 filed on Jun. 22, 2023 is incorporated herein by reference.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

### REFERENCE SIGNS

1 balloon catheter
5 hub
6 fluid injection portion
10 shaft
13 guidewire lumen
15 distal-side shaft
16 proximal-side shaft
20 balloon
20d distal end of balloon
20p proximal end of balloon
201 proximal-side sleeve portion
201d distal end of proximal-side sleeve portion
201p proximal end of proximal-side sleeve portion
202 proximal-side tapered portion
202d distal end of proximal-side tapered portion
202p proximal end of proximal-side tapered portion
203 straight tube portion
203d distal end of straight tube portion
203p proximal end of straight tube portion
204 distal-side tapered portion
204d distal end of distal-side tapered portion
204p proximal end of distal-side tapered portion
205 distal-side sleeve portion
205d distal end of distal-side sleeve portion
205p proximal end of distal-side sleeve portion
21 first balloon
22 second balloon
40 guidewire tube
50 guidewire port
60 end tip member
70 radiopaque marker
80 covering tube
130 inner tubular member
131 opening
135 distal-side inner tubular member
135d distal end of distal-side inner tubular member
136 proximal-side inner tubular member
140 outer tubular member
400 tubular member group
410 first outer tubular member
420 second outer tubular member
430 third outer tubular member
A3 distal region
A4 proximal region
A5 outer-tubular-member fixed region
A6 member fixed region
C410 center of first outer tubular member
C420 center of second outer tubular member
C430 center of third outer tubular member
D10 distance between center of first outer tubular member and center of second outer tubular member
D11 distance between center of first outer tubular member and center of the third outer tubular member
D12 distance between outer surface of first outer tubular member and outer surface of second outer tubular member
D13 distance between outer surfaces of outer tubular members adjacent to each other
L2 length from distal end of balloon to proximal end of balloon

1. A balloon catheter comprising:
a shaft extending from a proximal side toward a distal side in a longitudinal direction; and
a plurality of balloons disposed at a distal portion of the shaft,
wherein the shaft includes an inner tubular member having a lumen, and a tubular member group including a plurality of outer tubular members each having a lumen, the plurality of outer tubular members being disposed on an outer side with respect to the inner tubular member and aligned in a circumferential direction of the inner tubular member,
wherein, in a cross-section perpendicular to the longitudinal direction, the tubular member group includes a first outer tubular member, a second outer tubular member disposed adjacent to the first outer tubular member in the circumferential direction of the inner tubular member, and a third outer tubular member disposed adjacent to the first outer tubular member in the circumferential direction of the inner tubular member and on a side opposite to the second outer tubular member, and
wherein, in a cross-section perpendicular to the longitudinal direction, the shaft includes a portion in which a distance between a center of the first outer tubular member and a center of the second outer tubular member is larger than a distance between a center of the first outer tubular member and a center of the third outer tubular member.

2. The balloon catheter according to claim 1,
wherein the shaft has a distal region, which is a region on a distal side, and a proximal region, which is a region located on a proximal side with respect to the distal region, and
wherein, in a cross-section in the proximal region and perpendicular to the longitudinal direction, a distance between a center of the first outer tubular member and a center of the second outer tubular member is larger than a distance between a center of the first outer tubular member and a center of the third outer tubular member.

3. The balloon catheter according to claim 2, wherein, in the longitudinal direction, a length of the proximal region is equal to or less than a length of the distal region.

4. The balloon catheter according to claim 2, wherein the inner tubular member has, in the proximal region, an opening between the outer tubular members adjacent to each other in the circumferential direction of the inner tubular member, the opening allowing a lumen of the inner tubular member to be in communication with a region outside the inner tubular member.

5. The balloon catheter according to claim 4, wherein the opening is located between the first outer tubular member and the second outer tubular member in the circumferential direction of the inner tubular member.

6. The balloon catheter according to claim 2, wherein, in a cross-section in the proximal region and perpendicular to the longitudinal direction, a distance between an outer surface of the first outer tubular member and an outer surface of the second outer tubular member is larger than an outer diameter of each of the outer tubular members constituting the tubular member group.

7. The balloon catheter according to claim 2, wherein, in the proximal region, the tubular member group includes at least one set of the outer tubular members in which the outer tubular members adjacent to each other in the circumferential direction of the inner tubular member and constituting the tubular member group are in contact with each other, and at least one set of the outer tubular members in which the outer tubular members adjacent to each other in the circumferential direction of the inner tubular member and constituting the tubular member group are not in contact with each other.

8. The balloon catheter according to claim 7, wherein, among the outer tubular members constituting the tubular member group, the outer tubular members adjacent to each other in the circumferential direction of the inner tubular member are not in contact with each other in the distal region.

9. The balloon catheter according to claim 2, wherein, in a cross-section in the distal region and perpendicular to the longitudinal direction, a distance between outer surfaces of the outer tubular members constituting the tubular member group, which are adjacent to each other in the circumferential direction of the inner tubular member, is smaller than an outer diameter of each of the outer tubular members.

10. The balloon catheter according to claim 2, wherein, in a cross-section in the distal region and perpendicular to the longitudinal direction, a distance between a center of the first outer tubular member and a center of the second outer tubular member is 90% or more and 110% or less of a distance between a center of the first outer tubular member and a center of the third outer tubular member.

11. The balloon catheter according to claim 1, wherein the tubular member group includes the outer tubular members extending in a direction inclined with respect to a direction in which the inner tubular member extends.

12. The balloon catheter according to claim 4, wherein, on a proximal side with respect to the opening, the outer tubular members constituting the tubular member group are disposed to surround the inner tubular member in the circumferential direction of the inner tubular member.

13. The balloon catheter according to claim 12,
wherein the balloon catheter has an outer-tubular-member fixed region, which is a region in which the outer tubular members constituting the tubular member group are fixed to each other, and
wherein the opening is arranged in the outer-tubular-member fixed region.

14. The balloon catheter according to claim 4, further comprising a covering tube,
wherein the balloon catheter has a member fixed region, which is a region in which the covering tube and each of the outer tubular members constituting the tubular member group are fixed to each other, and
wherein the opening is arranged in the member fixed region.

15. The balloon catheter according to claim 14,
wherein, on a distal side with respect to the member fixed region, the covering tube is disposed with the inner tubular member and the tubular member group being disposed in a lumen of the covering tube, and
wherein, on a proximal side with respect to the member fixed region, the covering tube is disposed without presence of the inner tubular member and the tubular member group.

16. The balloon catheter according to claim 14,
wherein, on a distal side with respect to the member fixed region, the inner tubular member and the tubular member group are disposed in a lumen of the covering tube, and
wherein, on a proximal side with respect to the member fixed region, the tubular member group is disposed in the lumen of the covering tube without presence of the inner tubular member.

17. The balloon catheter according to claim 1,
wherein a lumen of the inner tubular member is a guidewire lumen through which a guidewire is configured to be inserted, and
wherein lumens of the outer tubular members constituting the tubular member group are inflation lumens through which a fluid to be supplied to lumens of the balloons is configured to pass, the inflation lumens each being in communication with a corresponding one of lumens of the plurality of balloons.

18. The balloon catheter according to claim 17, further comprising: a guidewire tube having a lumen in communication with the guidewire lumen,
wherein the guidewire tube is disposed in a lumen of one of the balloons.

19. A method comprising dilating an aortic valve, deforming a bioprosthetic valve placed in a heart, or destroying the bioprosthetic valve, each being performed using the balloon catheter according to claim 1.
